(19) United States
(12) Patent Application Publication
(10) Pub. No.: US 2001/0047156 A1
(43) Pub. Date: Nov. 29, 2001

(54) Needlepoint safety slide assembly

(76) Inventor: Jerry W. Parker, Costa Mesa, CA (US)

Correspondence Address: JERRY W. PARKER, 2354 SANTA ANA AV. # 12, COSTA MESA, CA, 92627, US

(21) Appl. No.: 09/781,794

(22) Filed: Feb. 12, 2001

Related U.S. Application Data
(60) Provisional application No. 60/181,470, filed on Feb. 10, 2000.
(60) Provisional application No. 60/224,534, filed on Aug. 11, 2000.

(51) Int. Cl. A61M 5/00

(52) U.S. Cl. 604/198; 604/263

(57) ABSTRACT

A syringe needle protective device for protectively covering the needlepoint after use with one hand. The device has a member attached to a needle hub embodying a slot and locking tongue. The shaft that passes through the support member has a notch for the tongue to engage preventing reversal of the shaft, on the needle end of the shaft is a cylinder through which the needle passes and when deployed completely covers the needle point. On the opposite end of the shaft is a sleeve that fully encompasses the syringe barrel allowing deployment from any position around the barrel.

### RELATED APPLICATIONS

This application claims the benefit of provisional applications Ser. No. 60/181,470 filed Feb. 10, 2000, Ser. No. 60/224,534 Aug. 11, 2000 and Ser. No. 60/225,31D filed Aug. 15, 2000.

### FIELD OF THE INVENTION

This invention relates to single use syringe and in particular to a single use syringe having a slidable non-reversible protection member with a locking feature

### BACKGROUND OF THE INVENTION

Healthcare workers are exposed to hypodermic needles after being used for injection or withdrawal of fluids from patients. Many patients having infectious blood pathogen diseases such as aids and hepatitis pose a real danger of transmitting the disease or infection by way of accidental needle sticks to the healthcare worker. There is a growing need to protect the workers from these accidental potentially harmful sticks.

### SUMMARY OF INVENTION

In accordance with the invention is to provide a simple, foolproof protective device for hypodermic syringes. The device consists of a slidable, locking, non-reversible protective fixture that covers the needlepoint after use.

### BRIEF DESCRIPTION OF THE DRAWINGS

FIG. 1, is a sectional view showing the device in its fully deployed position.

FIG. 2, is a perspective view of the device installed on a conventional hypodermic syringe in its ready to use configuration.

### DESCRIPTION OF THE PREFERRED EMBODIMENT

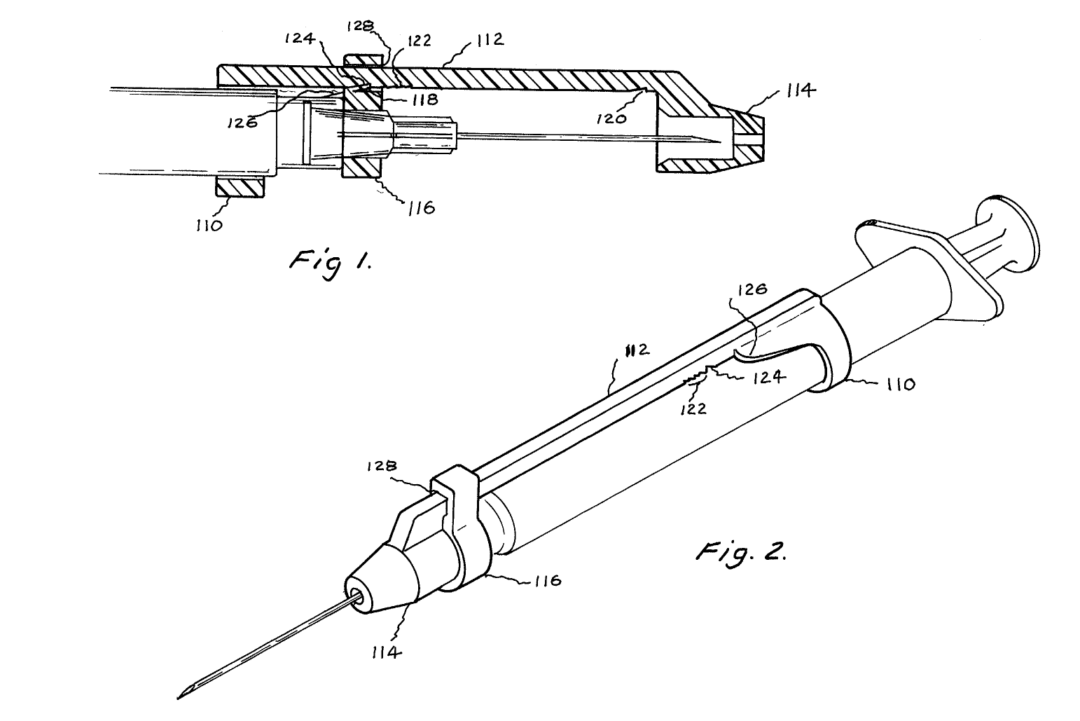

FIG. 1 The device consists of a wrap-around sleeve 110 connected to a square, “T” shaped, oval or flat shaft 112 of sufficient length to reach from the end of the syringe to the tip of the needle when fully deployed. At the needlepoint end of the shaft 112 is an elongated cylinder 114 through which the needle passes. In the undeployed position, the cylinder 114 rests against the needle hub. Around and securely attached to the needle hub is the supporting member 116. Embodied in the support is a flexible tongue 118 that allows the shaft 112 to freely slide through the slot 128 when being deployed. On the underside of the shaft 112 are notches. The notch 120 closest to the needle point end engages the flex tongue 118 when the device is in the undeployed position and serves as a detent to prevent accidental deployment. Notches 122 serve as a “feel warning” just prior to the flex tongue 118 engaging into notch 124 which locks the device in the fully deployed position and prevents the return of the shaft 112. The elongated cylinder 114 now completely covers the needle tip preventing accidental sticks. The sleeve 110 has an extended portion 126 along the shaft to serve as a stop when it comes to rest against the support member 116 in the fully deployed position positively locating the elongated cylinder correctly over the needle tip.

FIG. 2 shows the device properly installed on the syringe prior to use. The sleeve 110 that fully encompasses the syringe barrel allows user to deploy the device with one hand from any location around the syringe barrel.

It is understood that the embodiment described herein is only an example of the inventive needle protective device construction, and may be modified as desired within the scope of the appended claims.

What is claimed is:

1. A syringe needle protective device comprising:

2. A support member attached to a needle hub embodying a slot and flexible locking tongue. The syringe in claim 1. wherein has an elongated cylinder through which the needle passes attached to a notched shaft that slides through the support member to the locking position. The syringe in claim 1. herein has at the opposite end of the shaft is a wrap-around sieve fully encompassing the syringe barrel allowing the deployment of the device from any position around the syringe barrel.
